(19) United States
(12) Patent Application Publication
(10) Pub. No.: US 2025/0143907 A1
(43) Pub. Date: May 8, 2025

(54) Elastic neutral ankle foot orthosis

(71) Applicant: AMC Incorporated, Cheonan-si (KR)

(72) Inventor: Young In Hwang, Asan-si (KR)

(21) Appl. No.: 18/573,076

(22) PCT Filed: Dec. 13, 2023
(86) PCT No.: PCT/KR2023/020520

§ 371 Date: Jan. 22, 2025

(30) Foreign Application Priority Data
Mar. 29, 2023 (KR) 10-2023-0041153

(51) Int. Cl. A61F 5/01

(52) U.S. Cl. CPC A61F 5/0113

(57) ABSTRACT

Disclosed is an elastic neutral ankle-foot orthosis including: a forefoot immobilizer formed of rubber to cover a front of a foot; an elastic band connected to one end of the forefoot immobilizer; a calf band worn to be wrapped around the calf and having the elastic band attached thereto; and a knee retention band worn to be wrapped around a knee and having a top of the elastic band to be pulled and secured thereto.

### TECHNICAL FIELD

The present disclosure relates to an elastic neutral ankle-foot orthosis, and more specifically, to an elastic neutral ankle-foot orthosis capable of sufficiently assisting active muscle movement in patients with inversion or eversion during walking.

### BACKGROUND ART

Patients with paralysis due to cerebrovascular disease have weak muscle activity around the ankle joint due to paralysis of the nervous system, resulting in a gait pattern in which ankle dorsiflexion/lowering and eversion/adduction are not voluntarily performed when walking. In other words, due to the spastic paralysis of the plantar flexors, the initial stance is characterized by foot drop, where the posterior axis of the foot does not touch the ground and the foot is stepped on the ball of the foot or the tip of the foot.

The foot drop is a paralysis of the nervous system that results in reduced or absent muscle activity around the ankle, making it impossible to lift the foot. The foot drop is caused by paralysis of the central and peripheral nervous system, which prevents the tibialis anterior muscle around the ankle from working normally, especially in people with spastic paraplegia. In other words, to compensate for this, paralyzed people develop a circumduction gait, which is an inefficient gait that slows down walking speed and greatly increases energy expenditure. Ankle foot orthoses, which are worn as a physical aid to improve the gait in these patients, have become widely available.

Korean Patent Nos. 10-1682450, 10-1531203, and 10-1767996 disclose an ankle-foot orthosis having a foot support and a leg support formed of plastic.

These conventional ankle orthoses have a structure in which the foot support and the leg support are formed as a whole with an elastic material, or the foot support and the leg support formed with plastic are connected by a joint part, and the relative rotation angle is elastically supported by a spring.

However, because the support is formed of plastic in a three-dimensional manner, it is heavy and bulky, making it inconvenient for users to carry around. In addition, It is difficult for patients to actively perform gait training because the plastic foot support limits proprioception in the sole of the foot.

Meanwhile, inversion refers to a condition in which the talus and metatarsals are turned outward, meaning that the heel is turned inward than the tibia when the foot is viewed from above. Eversion refers to a condition in which the talus and metatarsals are turned inward, meaning that the heel is turned outward when viewed from above.

The human foot undergoes sequential occurrences of inversion and eversion during walking, and in most people, the degree of inversion and eversion remains small and within a certain range. However, the inversion or eversion becomes more severe, the foot walks in a tilted position, which reduces weight-bearing capacity and makes it more difficult to dissipate shock.

Korean Utility Model Registration No. 20-0202448 discloses an assistant of equinus drop foot. The disclosed conventional assistant of equinus drop foot includes: a first retention band having a fixation ring and an engagement ring riveted or sewn at one end for fixation to a knee joint of a lower leg; a second retention band having a fixation ring and a hook riveted or sewn at one end for fixation to a metatarsal of a foot; an elastic band portion connecting the first and second retention bands, the elastic band portion having a top joined to an engagement ring of the first retention band and a bottom joined to a hook of the second retention band, and having a fastener and an adjustment ring.

However, the conventional assistant of equinus drop foot has a problem in that the two retention band s fixed to the leg and foot compress the leg and foot strongly, causing discomfort, and conversely, it is easy to loosen when the bands are loosely fixed.

Furthermore, since the elastic band only applies an elastic force upward from the outer side of the metatarsal, it is difficult to fully support the gait of a patient with varus.

### DETAILED DESCRIPTION OF INVENTION

### Technical Problem

An object of the present disclosure is to provide an ankle orthosis which is easy to fabricate, carry, detach, and use, and whose elasticity is easily adjustable.

In particular, the object is to provide an elastic neutral ankle-foot orthosis capable of sufficiently supporting active movement of muscles during walking for patients with inversion or eversion.

### Technical Solution

An elastic neutral ankle-foot orthosis according to one form of the present disclosure for achieving the above object include: a forefoot immobilizer formed of rubber to cover a front of a foot; an elastic band connected to one end of the forefoot immobilizer; a calf band worn to be wrapped around the calf and having the elastic band attached thereto; and a knee retention band worn to be wrapped around a knee and having a top of the elastic band to be pulled and secured thereto.

The elastic band may include an outer elastic band integrally connected to an outer end of the forefoot immobilizer, and an inner elastic band integrally connected to an inner end of the forefoot immobilizer.

The elastic band may include a plurality of attachment straps provided at predetermined intervals on an inner surface thereof and attached and secured to the calf band.

The knee retention band may include: a knee brace formed of fabric and covering a knee area; a pair of retention straps provided at a top and a bottom of the knee brace and attached by wrapping around the top and bottom of the knee; and a plurality of engagement rings provided at a bottom of the knee brace and connected by pulling the top of the elastic band.

The elastic band may be formed so that elasticity gradually increases toward the top thereof.

The knee retention band may further include: a plastic bar inserted into the bottom of the knee brace 151 to be disposed below the knee; and a plurality of auxiliary wings joined to outer surfaces of the top and bottom of the knee brace to enhance retention.

An elastic neutral ankle-foot orthosis according to another aspect of the present disclosure includes: a toe receiver fitted to embrace toes and molded of a rubber material; a heel receiver connected to the toe receiver and molded to embrace a heel; an elastic band connected to a rear end of the toe receiver and pulled to be wrapped around the calf; and a knee band worn to be wrapped around the knee or below the knee and joined by pulling a top of the elastic band.

The elastic band may include: an upper elastic band connected to a upper rear end of the toe receiver so as to be wrapped around the calf, and joined to the knee band; and a lower elastic band connected to a rear lower end of the toe receiver and joined to an ankle area of the upper elastic band.

The upper elastic band may have a bottom joining portion joined by fusion to an upper joining portion extending from an upper rear end of the toe receiver, and the lower elastic band may have a bottom joining portion joined by fusion to a lower joining portion extending from a lower rear end of the toe receiver.

The heel receiver may include: a connecting strap connected between a rear end of the toe receiver and a front end of the heel receiver; and an ankle retention strap connected to one end of a top, fitting over the upper ankle, and coupled to the other end.

The knee band may include a Velcro tape and a Velcro attachment portion provided at one end and the other end, and the top of the elastic band may be connected to a middle of the knee band by a Velcro tape or by sewing.

The heel receiver may include a pair of band hangers that are formed in the form of rings on both sides of an upper portion of the heel receiver to hang the elastic band.

The elastic band may include: a lower joining portion joined to the upper rear end of the toe receiver by sewing or fusion; and an upper joining portion joined to a middle of the knee band by sewing or by a Velcro tape.

The elastic band may be pulled to be wrapped around the calf inwardly or outwardly.

The elastic band may be molded as an elastic band formed of neoprene rubber, silicone rubber, or synthetic rubber, and, and the knee band may be formed of a fabric material.

### Effect of Invention

According to the elastic neutral ankle-foot orthosis of the present disclosure described above, it is very easy to manufacture the ankle orthosis because it has a two-dimensional structure and is manufactured by sewing.

Additionally, the elastic neutral ankle-foot orthosis is lightweight and foldable for easy portability.

In addition, the elastic neutral ankle-foot orthosis is lightweight and may be secured to a leg to prevent movement during use, and are very easy for users to put on and take off

By adjusting the length of the elastic band, it is possible to control the force with which you lift the ball of your foot, and the elastic band itself may be formed of different materials with different modulus of elasticity. In addition, by adjusting the length of the elastic band, not only can the force that lifts the sole of the foot be adjusted, but the elastic band itself may be formed of various materials with different elastic coefficients.

In addition, it can sufficiently assist active muscle movement in patients with inversion or eversion when walking.

### BRIEF DESCRIPTION OF THE DRAWING

FIG. 1 is a perspective view showing a state in which an elastic neutral ankle-foot orthosis according to a first embodiment of the present disclosure is worn on a leg.

FIG. 2 is a development diagram schematically showing an elastic neutral ankle-foot orthosis according to the first embodiment of the present disclosure.

FIG. 3 is a partial perspective view showing the forefoot immobilizer and elastic band.

FIG. 4 is a conceptual diagram showing an elastic band according to a modified example of the present disclosure.

FIG. 5 is a conceptual diagram showing the elastic neutral ankle-foot orthosis according to the first embodiment of the present disclosure worn on the left leg in the first way.

FIG. 6 is a conceptual diagram showing the elastic neutral ankle-foot orthosis according to the first embodiment of the present disclosure worn on the left leg in a second manner.

FIG. 7 is a conceptual diagram showing the elastic neutral ankle-foot orthosis according to the first embodiment of the present disclosure worn on the left leg in a third way.

FIG. 8 is a conceptual diagram showing an elastic neutral ankle-foot orthosis according to a modified example of the present disclosure worn on the left leg.

FIG. 9 is a developed view showing the knee retention band and calf band of an elastic neutral ankle-foot orthosis according to another modification of the present disclosure.

FIG. 10 is a conceptual diagram showing the elastic neutral ankle-foot orthosis of FIG. 9 worn on the left leg in a third way.

FIG. 11 is a perspective view showing a state in which the elastic neutral ankle-foot orthosis according to the second embodiment of the present disclosure is worn on the leg.

FIG. 12 is a perspective view showing the elastic neutral ankle-foot orthosis of FIG. 11 as seen from the front outside.

FIG. 13 is a top view (a) and a bottom view (b) showing the connection structure of the forefoot mounting portion and the heel receiver.

FIG. 14 is a right side view (a) and left side view (b) showing the connection structure of the forefoot mounting portion and the heel receiver.

FIG. 15 is a developed view (a) of the upper elastic band connected to the knee band and a developed view (b) of the lower elastic band.

FIGS. 16 and 17 are perspective views showing a state in which an elastic neutral ankle-foot orthosis according to a modified example of the second embodiment is worn on a leg.

### BEST MODE FOR CARRYING OUT THE INVENTION

Since the present disclosure may be modified in various ways and can have various embodiments, specific embodiments will be exemplified and explained in detail in the detailed description. However, this is not intended to limit the present disclosure to the particular exemplary embodiments, and it should be noted that the present disclosure is intended to include all variations, equivalents, and substitutions that are included in the technical scope of the idea of the present disclosure.

The terms and expressions used in the present disclosure are used only for the purpose of illustrating particular embodiments, and are not intended to limit the present disclosure. A singular representation may include a plural representation unless it represents a definitely different meaning from the context. It should be noted that the terms “include” or “have” as used in the present disclosure are intended to denote the existence of any features, numerical values, steps, operations, constituent elements, parts, and combinations thereof described in the specification, but are not intended to preliminarily exclude the possibility of existence or addition of any one or more other features, numerical values, steps, operations, constituent elements, parts, and combinations thereof.

Hereinafter, preferred embodiments of the present disclosure will be described in detail with reference to the accompanying drawings. At this time, it should be note that in the accompanying drawings, it should be noted that the same elements will be designated by the same reference numerals, wherever possible. Also, detailed description of well-known related structures or functions will be omitted when it is deemed that such description will cause ambiguous interpretation of the present disclosure. For the same reason, some elements are exaggerated, omitted, or schematically shown in the accompanying drawings.

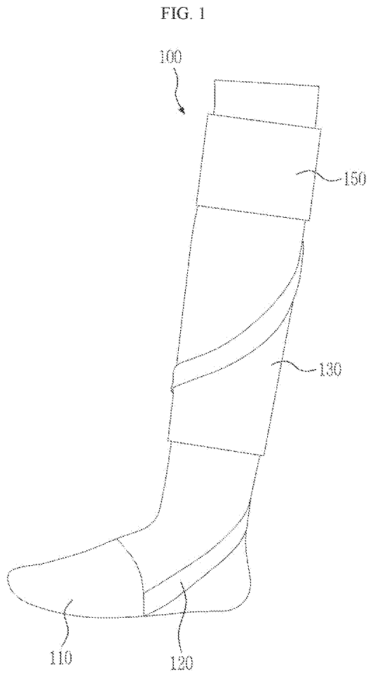

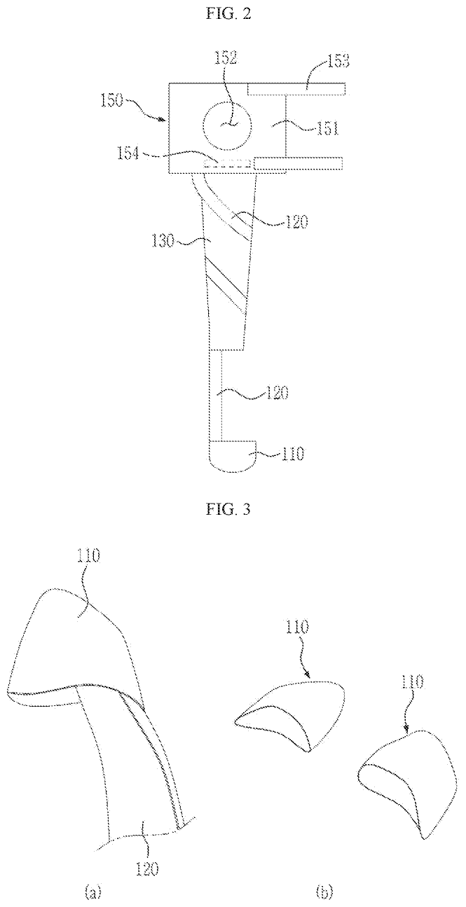

FIG. 1 is a perspective view showing a state in which an elastic neutral ankle-foot orthosis according to a first embodiment of the present disclosure is worn on a leg, FIG. 2 is a developed view schematically showing the elastic neutral ankle-foot orthosis according to the first embodiment of the present disclosure, and FIG. 3 is a partial perspective view showing a forefoot immobilizer and an elastic band.

An elastic neutral ankle-foot orthosis 100 of the present disclosure includes a forefoot immobilizer 110 formed of rubber to embrace the front of a foot, an elastic band 120 connected to one end of the forefoot immobilizer, a calf band 130 worn to be wrapped around a calf and allowing the elastic band to be attached thereto, and a knee retention band 150 worn to be wrapped around a knee and secured by pulling a top of the elastic band.

The forefoot immobilizer 110 is formed of silicone rubber and may be three-dimensionally molded to wrap around the front of the foot. As shown in FIG. 3 (b), the forefoot immobilizer 110 may be manufactured in such a way that one worn on the left foot and one worn on the right foot are symmetrical to each other. A length of the forefoot immobilizer 110 in a front-back direction may be longer on an outside than on an inside.

The elastic band 120 may be integrally formed while connected to one end of the forefoot immobilizer 110. The elastic band 120 may be formed of a silicone rubber material like the forefoot immobilizer 110, but may be molded integrally with the forefoot immobilizer 110 in a rubber material more flexible than a material of the forefoot lock 110. The elastic band 120 may be connected to an outer rear end or inner rear end of the forefoot immobilizer 110 to assist the walking of a patent with eversion or inversion, preventing the foot from turning inward or outward.

The calf band 130 may be formed of fabric in a shape of a circular tube so as to be worn to be wrapped around the calf. The calf band 130 may be worn to gently compress the calf, and as the elastic band 120 is attached to the calf band 130, it is possible to prevent the elastic band 120 formed of rubber from directly contacting the skin of the leg.

As shown in FIGS. 1 and 2, the elastic band 120 may be wrapped around the calf band 130 for a tight fit. The top of the elastic band 120 may be detachably joined to the knee retention band 150.

The knee retention band 150 may be worn to be wrapped around the knee and secured by a plurality of straps. A bottom of the elastic band 120 may be connected to one side of the forefoot immobilizer 110, and the top of the elastic band 120 may be pulled around the calf and joined and secured to the knee support band 150.

The calf band 130 may be joined to an inner surface of the knee retention band 150 at an upper front portion thereof. Since the calf band 130 is connected to the knee retention band 150, when the elastic band 120 is attached to the calf band 130, the elastic band 120 worn on the calf may be prevented from being pulled down by the calf band 130.

In the case of FIG. 1, a patient with inversion is wearing the elastic neutral ankle-foot orthosis 100 on a left leg, with the elastic band 120 that is connected to the left rear end of the forefoot immobilizer 110, wrapped around the calf toward the heel, and connected to the knee retention band 150.

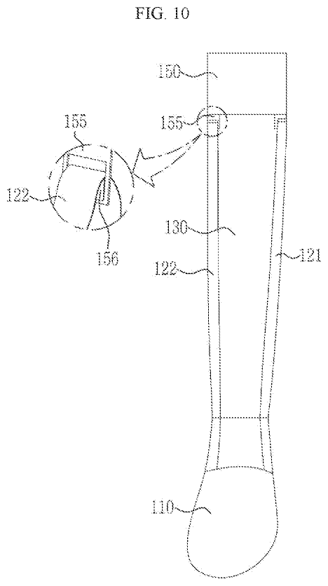

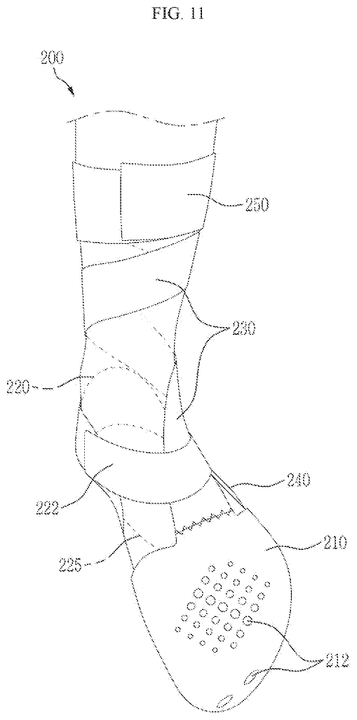

As shown in FIGS. 5 to 10, the elastic band 120 may include an outer elastic band 121 integrally connected to an outer end of the forefoot immobilizer 110, and an inner elastic band 122 integrally connected to an inner end of the forefoot immobilizer 110.

By simultaneously connecting the outer elastic band 121 and the inner elastic band 122 to the left and right sides of the foot forefoot immobilizer 110, it is possible to adjust a winding direction and pulling elastic force of the two elastic bands so that a patient with a varus or valgus can selectively use the elastic neutral ankle-foot orthosis 100 on the left leg or the right leg.

The outer elastic band 121 may be integrally connected to the outer rear end of the forefoot immobilizer 110, and may be pulled to fit tightly against the outer or inner side of the calf or may be wound clockwise or counterclockwise around the calf. The inner elastic band 122 may be integrally connected to the inner rear end of the forefoot immobilizer 110, and may be pulled to fit against the outer or inner side of the calf or may be wound clockwise or counterclockwise around the calf.

FIGS. 5 to 7 show a state in which the elastic neutral ankle-foot orthosis 100 having both the outer elastic band 121 and the inner elastic band 122 is wom on the left leg.

FIG. 5 shows a case where the outer elastic band 121 is wound counterclockwise and the inner elastic band 122 is wound clockwise when the left foot is viewed from above.

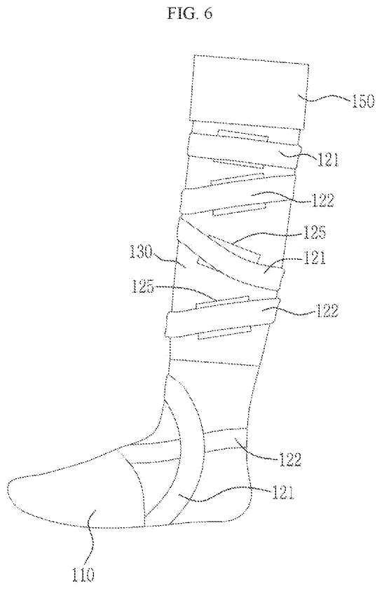

FIG. 6 shows a case where the outer elastic band 121 is wound clockwise and the inner elastic band 122 is wound counterclockwise when the left foot is viewed from above.

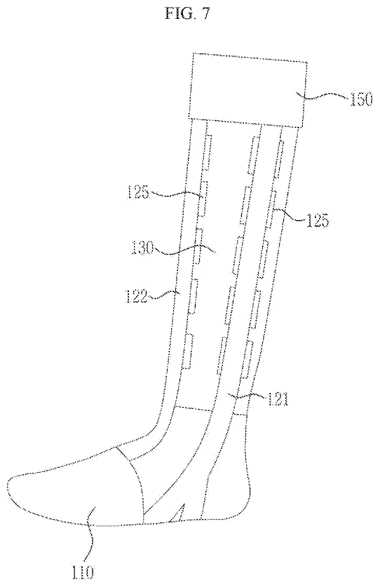

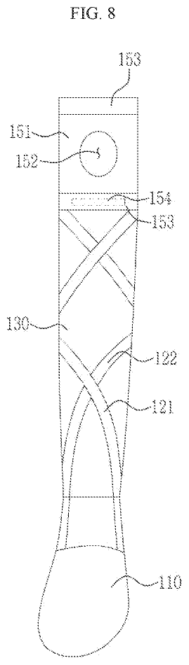

FIG. 7 shows a case where the outer elastic band 121 and the inner elastic band 122 are not wrapped around the calf, but are simply pulled upward to be connected to the knee retention band 150.

As shown in FIGS. 5 to 7, the elastic band 120 may include a plurality of attachment straps 125 provided at predetermined intervals on an inner surface thereof and attached to and secured to the calf band 130.

Each attachment strap 125 is formed in a rectangular shape with a predetermined length and width and may be sewn or glued to the inner surface of the elastic band 120. Five or more attachment straps 125 may be arranged at predetermined intervals on the inner surface of the elastic band 120. When the elastic band 120 stretches as the elastic band 120 is pulled, the attachment straps 125 may be sequentially attached to the calf band 130 to secure the elastic band 120. To this end, each attachment strap 125 may be provided with a Velcro tape, and the calf band 130 may be formed of a fabric material to which the Velcro tape may be detachably attached.

As shown in FIGS. 2, 9, and 10, the knee retention band 150 may include a knee brace 151 formed of a fabric material wrapping around a knee area; a pair of retention straps 153 provided at the top and bottom of the knee brace and attached by wrapping around the top and bottom of the knee; and a plurality of engagement rings 155 provided at the bottom of the knee brace to connect to the top of the elastic band 120 by pulling the top of the elastic band 120.

The knee brace 151 may be formed of a fabric material in an approximately rectangular shape and may be fitted to embrace the knee area. A knee hole 152 under which the patella is placed may be formed at a central portion of the knee brace 151.

To fix the knee brace 151, the pair of retention straps 153 may be joined to the top and bottom of the knee brace 151. Each retention strap 153 may be formed in the shape of an elongated band and may be provided with a Velcro tape sewn and attached to an inner surface thereof.

Four or more engagement rings 155 may be provided at predetermined intervals at the bottom of the knee brace 151. The engagement ring 155 may be connected by pulling the top of the elastic band 120 and making a knot.

The elastic band 120 may be pulled to be attached to the calf, and depending on the angle or number of turns or angle to be wrapped around the leg, the top of the elastic band 120 may be connected to an engagement ring 155 at a suitable location.

Each engagement ring 155 may further include an attachment portion 156 to which an attachment strap 125 of the elastic band 120 is attached. As a result, an elastic force of the elastic band 120 may be adjusted by pulling the elastic band 120 to attach one of the plurality of attachment straps 125 to the attachment portion 156. A portion remaining at the rear end of the elastic band 120 may be organized by inserting the portion into the engagement ring 155.

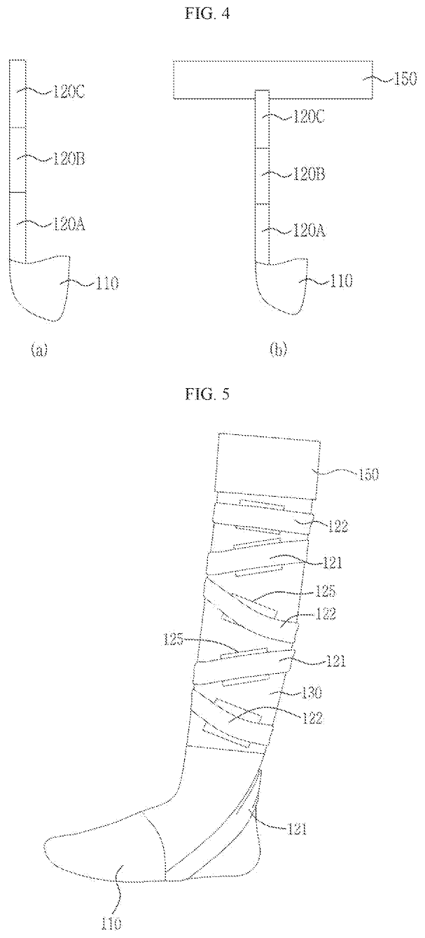

Meanwhile, as shown in FIG. 4, the elastic band 120 may be formed so that the elastic force thereof gradually increases toward the top.

That is, the elastic band 120 may be formed of a material whose elastic modulus gradually increases from portion A 120A through portion B 120B to portion C 120C. As a result, it is easier and more accurate to adjust the tension of the elastic band 120 as the elastic band 120 is pulled and connected to the knee retention band 150.

As shown in FIGS. 2 and 8, the knee retention band 150 may further include a plastic bar 154 inserted into the bottom of the knee brace 151 to be disposed below the knee.

The plastic bar 154 may be inserted into and fixed to the bottom of the knee brace 151 to support the patella below the knee hole 152. The plastic bar 154 may fix the knee by pushing the knee upward. As a result, the plastic bar 154 may increase the retention of the knee retention band 150, as well as make it easier for a person with weak knee cartilage to walk.

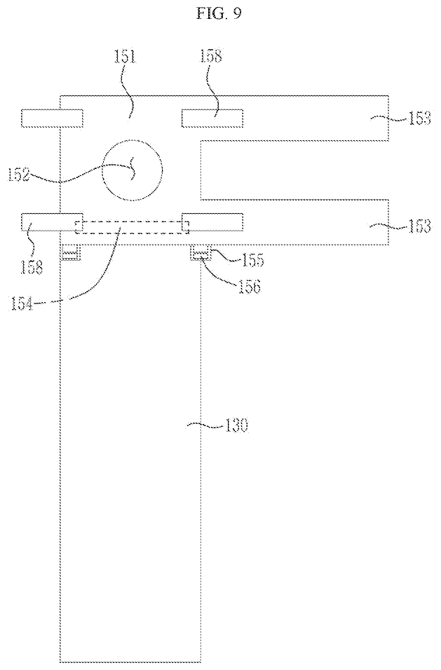

As shown in FIG. 9, the knee retention band 150 may further include a plurality of auxiliary wings 158 that are joined to the outer surfaces of the top and bottom of the knee brace 151 to enhance retention.

Four auxiliary wings 158 may be attached to the top, bottom, left and right sides of the knee brace 158. The auxiliary wings 158 may be formed of fabric in a rectangular band shape and sewn to one side of the knee brace 158.

The retention strap 153 may be wrapped around by the plurality of auxiliary wings 158, further enhancing the retention for securing the knee brace 151.

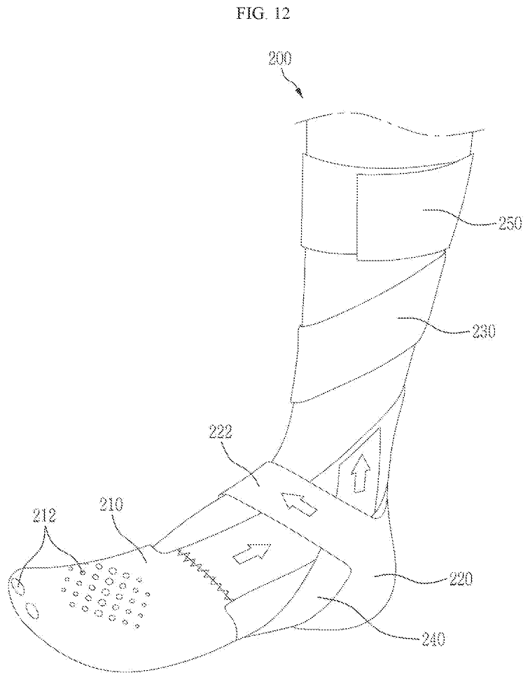

FIG. 11 is a perspective view showing a state in which an elastic neutral ankle-foot orthosis according to a second embodiment of the present disclosure is worn on a leg, and FIG. 12 is a perspective view showing the elastic neutral ankle-foot orthosis of FIG. 11 from an outward anterior perspective.

As shown in FIGS. 11 and 12, an elastic neutral ankle-foot orthosis 200 according to the second embodiment of the present invention: a toe receiver 210 fitted to embrace toes and molded of a rubber material; a heel receiver 220 connected to the toe receiver and molded to embrace the heel; an elastic band 230 connected to a rear end of the toe receiver and pulled to be wrapped around the calf; and a knee band 250 worn to be wrapped around the knee or below the knee and joined by pulling a top of the elastic band.

The toe receiver 210 may be manufactured to be three-dimensionally molded to embrace not only the toes but also the front half of the foot. The toe receiver 210 may be integrally formed of a rubber material such as neoprene or silicone.

The toe receiver 210 may include a plurality of ventilation holes 212 formed in an instep area or toe area. 10 ventilation holes 212 or more may be formed through the instep area, and may be formed in a circular or elongated shape. The ventilation holes 212 may formed in a variety of sizes. Two to three ventilation holes 212 may be formed in an oval or long hole shape in the toe area.

The heel receiver 220 may be three-dimensionally molded to embrace the heel, and may be connected to the toe receiver 210 by a strap or band.

The elastic band 230, which is a band formed of an easily elastically deformable rubber material, may be formed of neoprene rubber, silicone rubber, or synthetic rubber, and may include a highly elastic fabric material coated with silicone rubber.

The elastic band 230 may be connected to a rear end of the toe receiver 210 and may be pulled inward or outward to be wrapped around a calf. That is, depending on whether a user is a patient with inversion or eversion, the user may choose to wrap the elastic band 230 around the calf inward or outward. The top of the elastic band 230 may be joined to the middle of the knee band 250.

The knee attachment band 250 may be formed of a fabric material in a band shape so as to be worn to be wrapped around the knee or below the knee. While joined to the top of the elastic band 230 and pulling the elastic band 230 tightly, the knee band 250 may be secured by being wrapped around the knee or below the knee.

The elastic band 230 may be connected to an upper rear end of the toe receiver 210 so as to be wrapped around the calf, and may be joined at a top thereof to the knee band 250. A bottom of the elastic band 230 may be sewn, fused, or bonded with an adhesive at an instep position of the toe receiver 210. Thus, this elastic band 230 may be referred to as an upper elastic band 230.

As shown in FIG. 12, the elastic band may further include a lower elastic band 240 connected to a lower rear end of the toe receiver 210 and joined to an ankle area of the upper elastic band 230. A bottom of the lower elastic band 240 may be sewn, fused, or bonded with an adhesive to the sole of the toe receiver 210. The lower elastic band 240 may be pulled to pass a malleolus area, so that the top thereof is selectively joined to the ankle area of the upper elastic band 230.

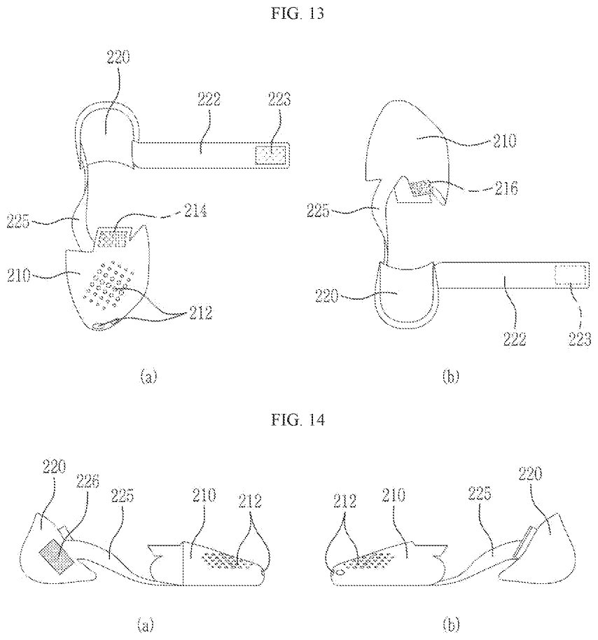

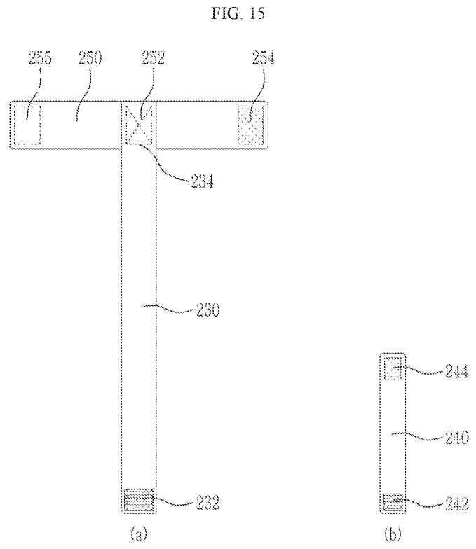

FIG. 13 is a top view (a) and a bottom view (b) illustrating the connection structure of a forefoot mount and a heel receiver, FIG. 14 is a right side view (a) and a left side view (b) illustrating the connection structure of the forefoot mount and the heel receiver, and FIG. 15 is a deployment view (a) of an upper elastic band and a deployment view (b) of a lower elastic band connected to a knee band.

As shown in FIGS. 13 and 15, an upper elastic band 230 may have a bottom joining portion 232 joined by fusion to an upper joining portion 214 extending from an upper rear end of a toe receiver 210, and a lower elastic band 240 may have a bottom joining portion 242 joined by fusion to a lower joining portion 216 extending from a lower rear end of the toe receiver 210.

As shown in FIGS. 13 and 15, the toe receiver 210 may include the upper joining portion 214 extending rearward from the upper rear end and the lower joining portion 216 extending rearward from the lower rear end. The lower joining portion 232 of the upper elastic band 230 may be joined by fusion to a bottom surface of the upper joining portion 214. The lower joining portion 242 of the lower elastic band 240 may be joined by fusion to a top surface of the lower joining portion 216. In other words, each of the upper elastic band 230 and the lower elastic band 240 may be joined by fusion to an inner peripheral surface of the rear end of the toe receiver 210.

As shown in FIG. 13, a heel receiver 220 may include a connecting strap 225 connected between a rear end of the toe receiver 210 and a front end of the heel receiver 220, and an ankle retention strap 222 connected to one end of the top, fitting over the upper ankle, and coupled to the other end.

The connection strap 225 may be formed of the same rubber material as that of the toe receiver 210 or the heel receiver 220. One end of the connection strap 225 may be connected to the lower rear end of the toe receiver 210, next to the lower joining portion 216. The other end of the connection strap 225 may be connected to one inner surface of the heel receiver 220.

One end of the ankle retention strap 222 is connected to the other inner surface of the heel receiver 220, and the other end may be provided with a Velcro tape 223.

As shown in FIG. 14, a Velcro attachment portion 226 is provided on one outer surface of the heel receiver 220, so that the Velcro tape 223 of the ankle retention strap 222 may be selectively attached.

As shown in FIGS. 11 and 12, the upper elastic band 230 may be pulled to be wrapped around the calf, the lower elastic band 240 may be pulled to attach a top portion thereof to the upper elastic band 230, and then the ankle strap 222 may be pulled over the ankle for a tight fit and secured by attaching the Velcro tape 223 to the Velcro attachment portion 226. As a result, the ankle retention strap 222 not only holds the heel receiver 220 from dislodging, but also may fix the upper elastic band 230 and the lower elastic band 240 by pressing the bands from above.

As shown in FIG. 15 (a), the knee band 250 may include a Velcro tape 254 and a Velcro attachment portion 255 at one end and the other end, and a top of the elastic band 230 may be joined by Velcro tape or sewn to the middle of the lap band 250.

The knee band 250 formed of a fabric material may be formed in the shape of an elongated rectangular band with a thickness of about 1 mm. The Velcro tape 254 may be fused, bonded, or sewn to the front of one end of the knee band 250, and the Velcro attachment portion 255, to which the Velcro tape 254 is joined, may be fused, bonded, or sewn to the back of the other end of the knee mounting strap 250.

The front of the lower part of the elastic band 230 is provided with a lower joining portion 232 joined by fusion to the upper joining portion 214 of the toe receiver 210, and the upper joining portion 234 of the elastic band 230 may be joined by sewing to a joining portion 252 of the knee band 250. That is, the lower joint portion 232 of the elastic band 230 and the joining portion 252 of the knee band 250 represent overlapping sewn areas.

In addition, as shown in FIG. 15 (b), the lower elastic band 240 may have a lower joining portion 242 provided at the bottom of a rear portion and fused to the lower joining portion 216 of the toe receiver 210, and an upper Velcro portion 244 provided at the front of an upper portion thereof and attached to the middle of the upper elastic band 230.

The lower elastic band 240 may be formed to have a smaller width than a width of the upper elastic band 230 and a length less than ⅓ of a length of the upper elastic band 230.

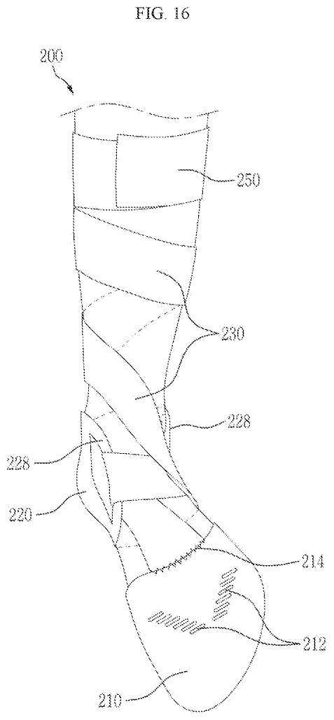

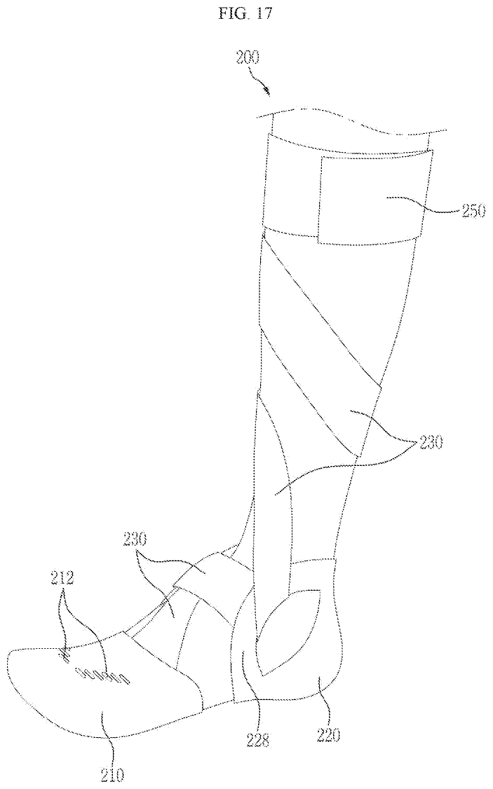

FIGS. 16 and 17 are perspective views showing a state in which an elastic neutral ankle-foot orthosis according to a modified example of the second embodiment is worn on a leg. This modified example differs from the second embodiment in that there is no lower elastic band and, instead of a connecting strap, the upper elastic band is wrapped around the calf while pulling the heel receiver up to secure.

That is, the elastic neutral ankle-foot orthosis 200 according to the modified example of the second embodiment includes a toe receiver 210, a heel receiver 220, an elastic band 230, and a knee band 250.

The heel receiver 220 may include a pair of band hangers 228, formed in the form of rings on both sides of the upper portion of the heel receiver to hang the elastic band 230.

In the modified example of FIG. 16, the bottom of the elastic band 230 is shown as joined by sewing to the upper joining portion 214 of the toe receiver 210, but it may also be fused or bonded with an adhesive.

The pair of band hangers 228 may be integrally formed in the shape of rings to support the elastic band 230 and have holes on both upper sides of the heel receiver 220 through which the elastic band 230 can pass.

In FIGS. 16 and 17, the elastic neutral ankle-foot orthosis 200 is shown wrapped inwardly around the left leg, wherein the elastic band 230 may be wrapped by passing sequentially through an inner band hanger 228 and an outer band hanger 228, from bottom to top, and then being pulled inwardly around the calf. Therefore, the elastic band 230 may not only exert a pulling force on to the inner or outer side of the ankle, but may also serve to pull and secure the heel receiver 220 toward the upper ankle.

Referring to FIG. 15, the elastic band 230 may include a bottom joining portion 232 joined by sewing or fusion to a rear end of the toe receiver 210, and a top joining portion 234 joined by sewing or by a Velcro tape to a middle of the knee band 250.

As described above, the lower joining portion 232 of the elastic band 230 may be joined to a rear upper joining portion 214 of the toe receiver 210 by sewing, fusion, or adhesive.

Additionally, the upper joining portion 234 of the elastic band 230 may be joined to the middle of the knee band 250 by sewing, joined by Velcro tape, or joined by a separate engagement ring so that a length of the upper joining portion is adjustable.

The elastic band 230 may be pulled to be wrapped around the calf inwardly or outwardly.

In the second embodiment of FIG. 11, the upper elastic band 230 is wrapped around the calf outwardly, but it may also be wrapped inwardly. When the upper elastic band 230 is wrapped inwardly, the lower elastic band 240 may also be pulled inwardly and attached to a malleolus area on the inner side the upper elastic band 230.

In the modified example of FIG. 16, the elastic band 230 is wrapped inwardly around the calf, but it may also be wrapped outwardly. When the elastic band 230 is wrapped outwardly, the elastic band 230 may pass through the outer band hanger 228, pass through the inner band hanger 228, and then be pulled outward of the calf to be wrapped around the calf.

Therefore, the elastic neutral ankle-foot orthosis 200 may be worn by wrapping the elastic band in an appropriate direction according to a condition of an inversion patient or eversion patient.

While the embodiments of the present disclosure have been described above, it will be understood by those of ordinary skill in the art that various modifications and changes may be made therein through inclusion, alteration, removal or addition of elements without departing from the spirit and scope of the present disclosure as defined by the following claims.

1. An elastic neutral ankle-foot orthosis comprising:
a forefoot immobilizer formed of rubber to cover a front of a foot;
an elastic band connected to one end of the forefoot immobilizer;
a calf band worn to be wrapped around the calf and having the elastic band attached thereto; and
a knee retention band worn to be wrapped around a knee and having a top of the elastic band to be pulled and secured thereto.

2. The elastic neutral ankle-foot orthosis of claim 1, wherein the elastic band comprises an outer elastic band integrally connected to an outer end of the forefoot immobilizer, and an inner elastic band integrally connected to an inner end of the forefoot immobilizer.

3. The elastic neutral ankle-foot orthosis of claim 1, wherein the elastic band comprises a plurality of attachment straps provided at predetermined intervals on an inner surface thereof and attached and secured to the calf band.

4. The elastic neutral ankle-foot orthosis of claim 3, wherein the knee retention band comprises:
a knee brace formed of fabric and covering a knee area;
a pair of retention straps provided at a top and a bottom of the knee brace and attached by wrapping around the top and bottom of the knee; and
a plurality of engagement rings provided at a bottom of the knee brace and connected by pulling the top of the elastic band.

5. The elastic neutral ankle-foot orthosis of claim 1, wherein the elastic band is formed so that elasticity gradually increases toward the top thereof.

6. The elastic neutral ankle-foot orthosis of claim 4, wherein the knee retention band further comprises:
a plastic bar inserted into the bottom of the knee brace 151 to be disposed below the knee; and
a plurality of auxiliary wings joined to outer surfaces of the top and bottom of the knee brace to enhance retention.

7. An elastic neutral ankle-foot orthosis comprising:
a toe receiver fitted to embrace toes and molded of a rubber material;
a heel receiver connected to the toe receiver and molded to embrace a heel;
an elastic band connected to a rear end of the toe receiver and pulled to be wrapped around the calf; and
a knee band worn to be wrapped around the knee or below the knee and joined by pulling a top of the elastic band.

8. The elastic neutral ankle-foot orthosis of claim 7, wherein the elastic band comprises:
an upper elastic band connected to an upper rear end of the toe receiver so as to be wrapped around the calf, and joined to the knee band; and
a lower elastic band connected to a rear lower end of the toe receiver and joined to an ankle area of the upper elastic band.

9. The elastic neutral ankle-foot orthosis of claim 8,
wherein the upper elastic band has a bottom joining portion joined by fusion to an upper joining portion extending from an upper rear end of the toe receiver, and
wherein the lower elastic band has a bottom joining portion joined by fusion to a lower joining portion extending from a lower rear end of the toe receiver.

10. The elastic neutral ankle-foot orthosis of claim 8, wherein the heel receiver comprises:
a connecting strap connected between a rear end of the toe receiver and a front end of the heel receiver; and
an ankle retention strap connected to one end of a top, fitting over the upper ankle, and coupled to the other end.

11. The elastic neutral ankle-foot orthosis of claim 7,
wherein the knee band comprises a Velcro tape and a Velcro attachment portion provided at one end and the other end, and
wherein the top of the elastic band is connected to a middle of the knee band by a Velcro tape or by sewing.

12. The elastic neutral ankle-foot orthosis of claim 7, wherein the heel receiver comprises a pair of band hangers that are formed in the form of rings on both sides of an upper portion of the heel receiver to hang the elastic band.

13. The elastic neutral ankle-foot orthosis of claim 12, wherein the elastic band comprises:
a lower joining portion joined to the upper rear end of the toe receiver by sewing or fusion; and
an upper joining portion joined to a middle of the knee band by sewing or by a Velcro tape.

14. The elastic neutral ankle-foot orthosis of claim 7, wherein the elastic band is pulled to be wrapped around the calf inwardly or outwardly.

15. The elastic neutral ankle-foot orthosis of claim 7,
wherein the elastic band is molded as an elastic band formed of neoprene rubber, silicone rubber, or synthetic rubber, and
wherein the knee band is formed of a fabric material.
